(19) United States
(12) Patent Application Publication
(10) Pub. No.: US 2003/0139768 A1
(43) Pub. Date: Jul. 24, 2003

(54) Roller milker device

(76) Inventors: Eugene F. Schrader, St. Louis, MO (US); David R. Swisher, St. Charles, MO (US); Daniel Peterson, Chicago, IL (US); Kurt Peterson, Mount Prospect, IL (US); James Hand, Glen Ellyn, IL (US); Douglas Elsie, Chicago, IL (US)

Correspondence Address: Greensfelder, Hemker & Gale, P.C., Suite 2000, 10 South Broadway, St. Louis, MO, 63102, US

(21) Appl. No.: 10/206,555

(22) Filed: Jul. 26, 2002

Related U.S. Application Data
(60) Provisional application No. 60/350,115, filed on Jan. 18, 2002.

(51) Int. Cl. A61B 17/28

(52) U.S. Cl. 606/209

(57) ABSTRACT

A milking device having a flexible, unitary body for securing and compressing patient tubing is disclosed comprising first and second portions which are tapered from one another and define a conduit that communicates with a channel formed on one side of the device and an opposing slot formed on the other side thereof. First and second portions further define first and second receptacles, respectively, each adapted to receive a roller disposed rotatably therein for engaging patient tubing. A pair of guides are provided at the entrance of the device in order to facilitate the axial advancement of patient tubing through the conduit of the body. Distal and proximal openings are formed at either end of the conduit to receive a portion of patient tubing inserted through the channel. The device may then be advanced longitudinally along the tubing as a user presses the two rollers together and engages the tubing while progressively evacuating the contents thereof into an appropriate collection site.

### BACKGROUND OF THE INVENTION

1. Field of the Invention

The present invention relates to medical devices, and more particularly to medical devices for milking patient tubing. More specifically, the present invention relates to a milking device for progressively clearing tube contents from patient tubing during a milking procedure.

2. Prior Art

Prior art medical devices that provide a means for clearing or dislodging the contents of patient tubing are well known in the art, such as devices used for stripping or milking. Devices used for stripping or milking procedures may include a pair of rollers that engage patient tubing therebetween in order to allow the user to clamp down on the tubing. The user then advances the device along the patient tubing such that tube contents are progressively cleared from the tubing. Typically, a stripping procedure requires the user to totally occlude the tubing, while the user in a milking procedure clamps down on the tubing, but does not totally occlude it.

For example, U.S. Pat. No. 3,847,370 to Engelsher discloses a tube servicing device comprising a strap-like frame with a pair of oppositely disposed flexible legs with each leg having an opposing roller capable of pinching, rolling contact with patient tubing. Other references, such as U.S. Pat. No. 4,164,223 to Munib, disclose an instrument for use in a stripping procedure comprising a pair of rollers mounted on shafts which are hinged together at one end, while the opposite end of one shaft forms a cylindrical handle and the other shaft an opposed thumb rest for moving the rollers into and out of engagement with the tubing. Similarly, U.S. Pat. Nos. 4,287,890; and 4,452,244 to Chin and Fogarty, respectively, disclose scissor-like instruments having opposed rollers mounted on each scissor portion for engaging and stripping patient tubing. Unfortunately, the above devices are provided with unguided rollers which may cause the device to run across the patient tubing thereby scoring and possibly tearing it.

Therefore, further improvements in the prior art are desirable for a device capable of simultaneously milking or stripping patient tubing while axially guiding the patient tubing as the-device is advanced along the length thereof in order to progressively expel the contents from the tubing.

### OBJECTS AND SUMMARY OF THE INVENTION

Accordingly, the primary object of the present invention is to provide a device for milking or stripping patient tubing.

Another object is to provide a device of the above character that includes at least a pair of opposed rollers capable of engaging patient tubing in continuous, rolling contact during the milking procedure.

Yet another object of the present invention is to provide a device of the above character having a flexible unitary body adapted to engage and axially guide patient tubing during the milking procedure.

A further object of the present invention is to provide a device of the above character having a channel adapted to laterally receive a portion of patient tubing therethrough during the milking procedure.

Yet another further object of the present invention is to provide a device of the above character having a flexible body that permits easy compression of the free ends of the device during the milking procedure.

These and other objects of the present invention are realized in the preferred embodiment of the present invention, described by way of example and not by way of limitation, which provides for a milking device for expelling the contents of patient tubing as the device axially guides patient tubing.

In brief summary, the present invention overcomes and substantially alleviates the deficiencies in the prior art by providing a milking device comprising a flexible unitary body defining first and second portions that collectively form a conduit adapted to receive patient tubing. The body further defines proximal and distal portions forming respective openings at opposing ends of the conduit. The first and second portions define a channel along one side and a slot along the other opposite side of the milking device. In the preferred embodiment, the distal end of each respective portion further defines opposing receptacles sized and shaped to receive a roller that is freely rotatable therein with one roller being in direct opposition with the other roller. However, in the alternative, three or more rollers may be utilized without departing from the scope of the present invention with the rollers in general opposition to one another. The body of the milking device is made from a flexible plastic material that permits the user to flex the distal end of first and second portions toward one another in order to compress patient tubing during the milking procedure. Further, the distal portion of the device defines opposing guides adapted to facilitate the axial guiding of patient tubing into the conduit of the body during the milking procedure.

In operation, the user laterally inserts the patient tubing through the channel and into the conduit such that the tubing is placed between the rollers and extends through the proximal and distal openings of the milking device. The user then grasps the first and second portions between his thumb and forefinger, respectively and flexes the distal end of the first and second portions toward one another such that the rollers properly engage and compress the tubing from the patient. As the milking device is axially advanced along the length of the patient tubing by the user, the guides in conjunction with the conduit of the body, axially guide the patient tubing as the contents therein are progressively dislodged from the tubing as the user maintains the compressive engagement therealong until all of the tubing contents are evacuated into a suitable collection site. Once the procedure is completed, the user removes the patient tubing from the milking device back through the channel.

Additional objects, advantages and novel features of the invention will be set forth in the description which follows, and will become apparent to those skilled in the art upon examination of the following more detailed description and drawings in which like elements of the invention are similarly numbered throughout.

### BRIEF DESCRIPTION OF THE DRAWINGS

FIG. 1 is a perspective view of a milking device according to the present invention;

FIG. 2 is a side view of the milking device according to the present invention;

FIG. 3 is an opposite side view of the milking device according to the present invention;

FIG. 4 is a cross-sectional view of the milking device taken along line 4-4 of FIG. 2 according to the present invention;

FIG. 5 is a cross-sectional view of the milking device taken along line 5-5 of FIG. 3 according to the present invention;

FIG. 6 is a top view of the milking device according to the present invention;

FIG. 7 is a bottom view of the milking device according to the present invention;

FIG. 8 is a perspective view of the milking device showing a pair of rollers according to the present invention; and

FIG. 9 is a partial exploded view of the milking device according to the present invention;

FIG. 10 is a cross-sectional view of the milking device engaged to patient tubing illustrating the milking procedure according to the present invention; and

FIG. 11 is a side view of an alternative embodiment of the milking device according to the present invention.

### DETAILED DESCRIPTION OF THE INVENTION

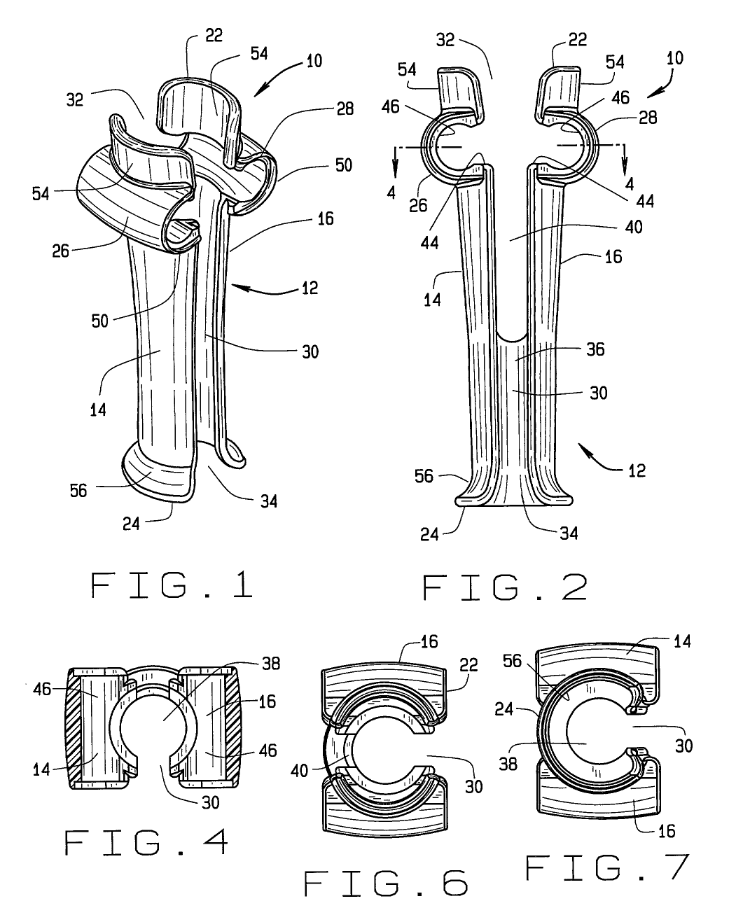

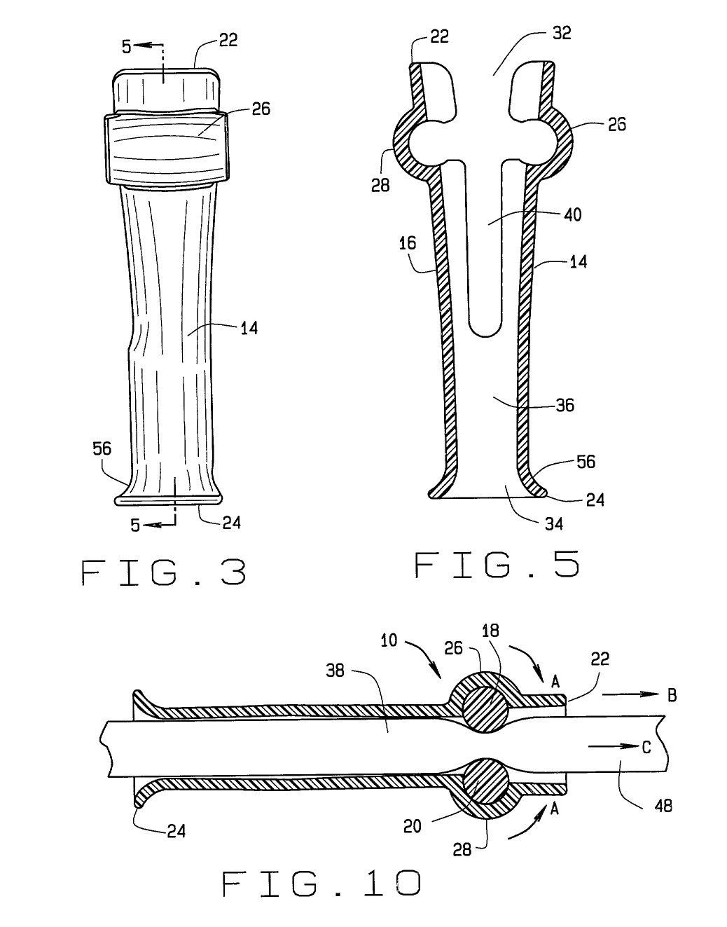

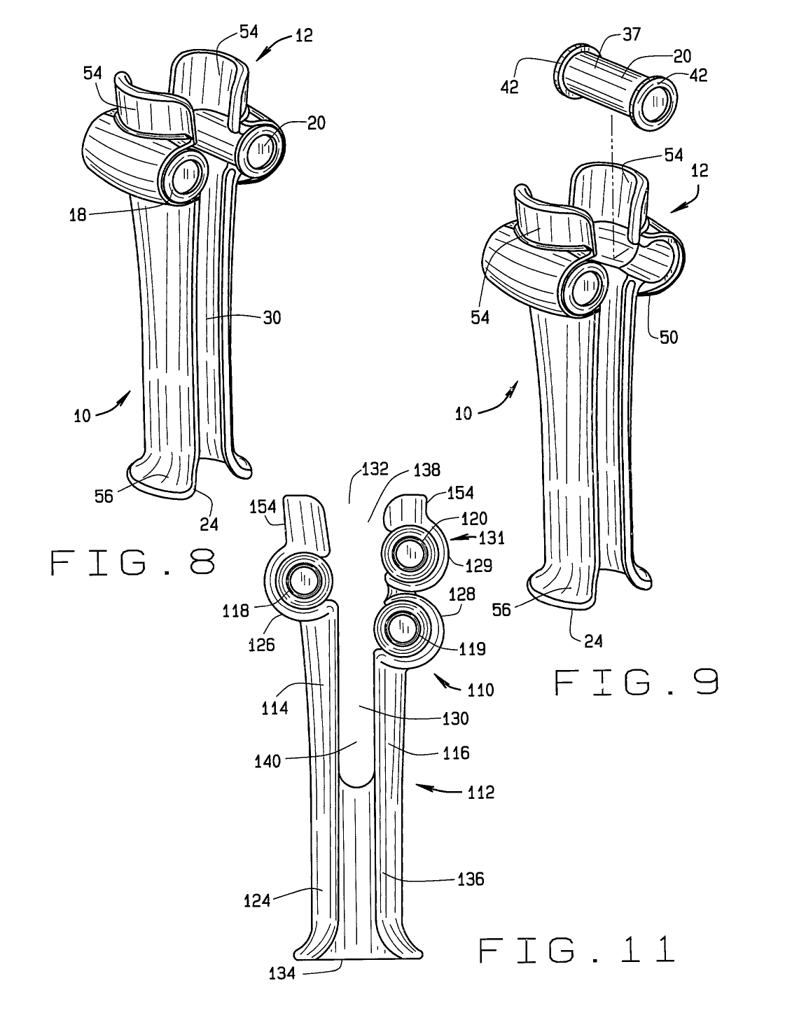

Referring to the drawings, the preferred embodiment of the milking device of the present invention for engaging and milking patient tubing is illustrated and generally indicated as 10 in FIG. 1. Milking device 10 comprises a flexible, unitary body 12 that includes opposing first and second portions 14 and 16 defining a distal portion 22 and a proximal portion 24. Referring to FIGS. 1, 2 and 5, first and second portions 14 and 16 collectively define a longitudinal channel 30 along an entire side of milking device 10 and a longitudinal slot 40 (FIG. 5) on the other side thereof that extends a distance from the distal portion 22 which both communicate with an interior conduit 38 formed between portions 14 and 16 of body 12. The conduit 38 includes distal and proximal openings 32 and 34 formed at opposite ends of conduit 38 for receiving a portion of patient tubing 48 (FIG. 10) therethrough during the milking procedure as shall be discussed in greater detail below. Referring specifically in FIG. 2, first and second portions 14 and 16 meet along proximal portion 24 by an integral portion 36 such that portions 14 and 16 are bifurcated and gradually taper away from one another as one proceeds from the proximal portion 24 to the distal portion 22 of milking device 10. In this tapered configuration, the inner diameter of the distal opening 32 is greater than the inner diameter of the proximal opening 34. Further, the tapered configuration permits first and second, portions 14 and 16 to be flexed toward one another when the user engages the rollers 18 and 20 with patient tubing 48 during the milking procedure.

As shown specifically in FIGS. 1, 2, 4, 8 and 9, first and second portions 14 and 16 define opposing receptacles 26 and 28, respectively. Receptacles 26 and 28 each include a curved segment 46 which is sized and shaped to securely engage a pair of rollers 18 and 20, respectively. Referring specifically to FIG. 9, rollers 18 and 20 each comprise a shaft 37 that forms opposing flanges 42 at each end thereof which are adapted to rotatably engage shoulders 50 formed at opposite ends of receptacles 26 and 28 when rollers 18 and 20 are secured therein. Once properly secured, rollers 18 and 20 freely rotate within the curved segments 46 of receptacles 26 and 28. Preferably, first and second receptacles are configured for snap fit engagement with rollers 18, 20.

Referring to FIGS. 5 and 6, distal portion 22 is formed adjacent distal opening 32 and defines a pair of guides 54 that facilitate the advancement of patient tubing 48 through conduit 38 of milking device 10. Guides 54 have a generally cup-shaped configuration adapted to facilitate advancement of patient tubing 48 through distal opening 32 during the milking procedure.

Referring to FIGS. 1, 3, and 7, proximal portion 24 defines an outwardly extending flange 56 which provides a gripping surface for the user. Flange 56 is configured such that the user may comfortably grasp and handle milking device 10 while permitting the distal portion 22 of first and second portions 14, 16 to be easily brought toward one another during the milking procedure and compress patient tubing 48.

Referring to FIG. 10, the operation of milking device 10 shall be discussed. In operation, the far end of patient tubing 48 is laterally inserted through channel 30 of milking device 10 such that the patient tubing 48 enters conduit 38 and is then inserted through both distal and proximal openings 32 and 34. In this arrangement, patient tubing 48 is in co-axial alignment with the conduit 38. The user then presses the distal portion 22 of first and second portions 14 and 16 together along arrows A with his thumb and forefinger in order to engage and compress patient tubing 48 between rollers 18 and 20 such that tubing 48 becomes partially occluded. Preferably, the user partially occludes patient tubing 48 by permitting rollers 18 and 20 to compress tubing 48 and expel tubing contents when milking device 10 is advanced in the direction of arrow B. While maintaining this engagement that compresses patient tubing 48, the user axially advances body 12 along the tubing 48 away from the patient until the contents thereof are expelled along the direction of arrow C into a suitable collection device, such as a chest drainage apparatus. However, the present invention also contemplates that milking device 10 can be used to totally occlude the patient tubing when device 10 is used in a stripping procedure. During the milking or stripping procedure, the conduit 38 in conjunction with guides 54 facilitate the axial advancement of patient tubing 48 through milking device 10 by maintaining the tubing 48 in proper alignment between rollers 18 and 20 so that tubing 48 does not disengage therefrom. This guiding feature of milking device 10 prevents the device 10 from running across patient tubing 48 which can score and tear it.

In one embodiment of the present invention, body 12 may have marking indicia, such as suitable language or directional arrow, that provides the user with the proper direction for advancing milking device 10 along patient tubing 48 during the milking operation, such direction being away from the patient and toward the collection device (not shown).

Preferably, milking device 10 is made from a suitable flexible plastic material that permits the user to easily flex the distal portion 22 inwardly when engaging patient tubing 48.

The present invention contemplates that the inner diameters of distal and proximal openings 32, 34 and conduit 38 are such that patient tubing 48 readily advances therethrough during the milking operation.

An alternative embodiment of the present invention is illustrated and generally indicated as 110 in FIG. 11. Milking device 110 comprises a similarly flexible body 112 that includes first and second portions 114 and 116, defining a distal portion 122 and a proximal portion 124. Further, first and second portions 114 and 116 collectively define a channel 130 on one side of milking device 110 and a slot 140 on the other side thereof which communicate with a conduit 138 formed between portions 114 and 116. The conduit 138 includes distal and proximal openings 132 and 134 formed at opposite ends thereof. The alternate embodiment differs from the preferred embodiment in that it includes a roller assembly 13 having receptacles 126, 128 and 129 which are arranged in general opposition to at least one other receptacle. Receptacles 126, 128 and 129 are adapted to rotatably secure rollers 118, 119 and 120, respectively. However, the present invention contemplates that at least two or more rollers be arranged to provide the means for compressing patient tubing 48.

It should be understood from the foregoing that, while particular embodiments of the invention have been illustrated and described, various modifications can be made thereto without departing from the spirit and scope of the invention. Therefore, it is not intended that the invention be limited by the specification; instead, the scope of the present invention is intended to be limited only by the-appended claims.

What is claimed is:

1. A medical device for expelling the contents of tubing comprising:
first and second portions, each of said first and second portions including at least one receptacle, a conduit defined between said first and second portions in communication with a distal opening and a proximal opening formed at opposite ends thereof;
a roller disposed within each of said receptacles, each of said rollers being freely rotatably within a respective receptacle and positioned in general opposition to one another,
wherein said first and second portions are flexible toward one another in order to engage the tubing between said rollers in a milking or stripping procedure such that said conduit facilitates the axial advancement of tubing therethrough.

2. The medical device according to claim 1, wherein each of said rollers comprises a cylindrical body defining flanges along opposite ends thereof.

3. The medical device according to claim 2, wherein each of said receptacles forms opposing shoulders adapted to rotatably receive a respective roller.

4. The medical device according to claim 3, wherein said flanges of said rollers are adapted to rotatably engage said shoulders of said receptacle.

5. The medical device according to claim 1, wherein said first and second portions are tapered away from one another.

6. The medical device according to claim 1, wherein a flange is formed adjacent said proximal opening, said flange adapted to provide a gripping surface.

7. The medical device according to claim 1, wherein a pair of guides are formed adjacent said distal opening, said guides facilitating the axial advancement of the tubing into said conduit.

8. The medical device according to claim 1, wherein each receptacle forms a curved segment, said curved segment being adapted to rotatably receive a respective roller therein.

9. The medical device according to claim 1, wherein said slot and said channel permit the first and second portions to be flexed toward one another.

10. The medical device according to claim 1, wherein the tubing is insertable into said conduit through said channel of the medical device.

11. The medical device according to claim 1, wherein a channel is formed between said first and second portions on one side of the medical device.

12. The medical device according to claim 1, wherein a slot is formed between first and second portions on the other side of the medical device.

13. A device for expelling contents from flexible tubing comprising:
a unitary body having at least a pair of receptacles, said body being formed of a flexible material and further including a proximal portion and a distal portion, said body defining a longitudinal channel extending along an entire length of said body from said distal portion to said proximal portion, said channel communicating with an interior conduit defined by said body, wherein said body is adapted to receive a length of flexible tubing within said interior conduit in coaxial relationship thereto by laterally passing said tubing through said channel and along said length of said body, said distal portion being bifurcated into opposing first and second portions, said receptacles each having a roller disposed therein, said first and second portions being adapted to be manipulated such that said rollers are drawn towards each other in approaching engagement.

14. The device according to claim 13 wherein said conduit facilitates axial advancement of said tubing through the device.

15. The device according to claim 14 wherein first and second portions are tapered, said distal portion of said body having a greater inner diameter than said proximal portion thereof.

16. The device according to claim 13 wherein each of said receptacles defines a shoulder at opposing ends thereof with each roller forming a flange at opposing ends thereof, said flanges being rotatably secured against each shoulder such that each of said rollers freely rotates with each of said receptacles.

17. The device according to claim 13 wherein said body is marked with indicia for instructing a user on a proper direction of advancement of said body during a milking procedure.

18. The device according to claim 13 wherein said distal portion defines opposing guides, said guides providing a means for facilitating advancement of tubing through said conduit.

19. A device for milking contents from flexible tubing, said device comprising:
a flexible body, said body defining a proximal portion and a distal portion and further defining a longitudinal channel extending along an entire length of said body from said distal portion to said proximal portion, said channel communicating with an interior conduit defined by said body, whereby said body being adapted to receive a length of flexible tubing within said interior conduit in coaxial relationship by laterally passing the tubing through said channel along said length of said body, said body further including a roller assembly comprising of at least a plurality of receptacles, each of said at least a plurality of receptacles having a roller rotatably engaged therein and in general opposition with at least one other receptacle, said roller assembly being adapted to be manipulated such that at least one receptacle is drawn toward another receptacle in approaching engagement.

20. The device according to claim 19 wherein an outwardly extending peripheral flange is formed at said proximal end of said body.

21. The device according to claim 20 wherein a shape of said hollow body is tapered, said distal end having a greater diameter than said proximal end.

22. The device according to claim 19 wherein said body is marked with indicia for instructing a user on a proper direction of advancement of said body during operation of the device.
